(19) United States
(12) Patent Application Publication
(10) Pub. No.: US 2023/0356009 A1
(43) Pub. Date: Nov. 9, 2023

(54) MASK DEVICE HAVING AIR CURTAIN FUNCTION

(71) Applicant: Hae Kon LEE, Cheongju-si (KR)

(72) Inventor: Hae Kon LEE, Cheongju-si (KR)

(21) Appl. No.: 17/428,317

(22) PCT Filed: Nov. 27, 2020
(86) PCT No.: PCT/KR2020/017141

§ 371 Date: Aug. 4, 2021

(30) Foreign Application Priority Data
Mar. 16, 2020 (KR) 10-2020-0031878

(51) Int. Cl. A62B 18/00; A61L 2/10; A62B 18/02; A62B 23/02

(52) U.S. Cl. CPC A62B 18/003; A61L 2/10; A62B 18/006; A62B 18/025; A62B 23/02

(57) ABSTRACT

Proposed is a mask device having an air curtain function according to the present invention. The mask device includes: a main body brought into tight contact with the vicinities of a user's nose and mouth; a respirator installed on a lower portion of the main body for the user's respiration; and a cover being combined with a front surface of the main body and covering the main body and the respirator. The respirator is configured to have a front-surface portion and a rear-surface portion. The respirator includes an inhalation-air supply portion provided in an upper portion thereof and an air curtain portion provided under the inhalation-air supply portion.

### TECHNICAL FIELD

The present invention relates to a mask device for blocking fine dust and for limiting the spread of a respiratory disease and, more particularly, to a mask device having an air curtain function, the mask device being configured to form an air curtain in the vicinity of a user's nose and mouth, thus blocking introduction of contaminated outside air and at the same time supplying fresh and clean air purified by a filter to the vicinity of the user's nose and mouth, and maintaining a comfortably worn state.

### BACKGROUND ART

Today, there is increasing likelihood that people will contract a respiratory disease due to exposure to a variety of environmental air pollutants, such as harmful gas, yellow dust, and fine dust. The problem of air pollution may be said to have originated with the Industrial Revolution, and air pollution has been growing with industrial development and urban development.

Air pollution is caused by solid and liquid particles and certain gases coming from natural phenomena such as a sandstorm in a desert or volcanic eruption and from man-made phenomena such as burning of fossil fuel in factories and vehicles.

Usually, natural air pollutants occur temporarily due to natural phenomena and do not become continuous threats to human respiratory health. However, man-made air pollutants result from burning of fuel in vehicles and airplanes, use of nuclear energy, chemical reactions, and chemical processes in factories. Among these, various air pollutants occurring when fuel is burnt have a continuous harmful effect on human respiratory health.

Carbon monoxide (CO), nitrogen dioxide (NO₂), and the like account for a high percentage of the air pollution. Particularly, sulfur dioxide (SO₂) reacts with other air pollutants and produces additional secondary pollutants. Air pollutants are categorized into gaseous materials, such as sulfurous acid gas or carbon monoxide, and particulate materials, such as dust particles.

In addition, the primary causes of air pollution are yellow dust, windblown dust, pollen, airborne bacteria, a forest fire, a volcanic eruption, and the like. Exposure to air pollution has many substantial effects on the health of humans.

In addition, air pollutants from a construction site, a place where sweeping takes place, a chemical manufacturing factory, a painting factory, a hospital, and the like are harmful to the health of humans.

Particularly, fine dust having a harmful effect on the human respiratory health is an airborne particulate matter of a diameter of 10 μm or less containing various components. This fine dust is an air pollutant that occurs when fossil fuel, such as coal or oil, is burnt. The fine dust is contained in gas discharged from a manufacturing factory, a vehicle, and the like.

The fine dust mainly contains carbon that is a combustion particle, organic hydrocarbon, nitrate, sulphate, and a harmful metal component and is usually associated with outbreaks of respiratory and cardiovascular diseases for which a death rate is increasing.

Accordingly, devices for eliminating fine dust have been developed. An example of these devices is disclosed in Korean Patent No. 10-1633878 titled “DUST COLLECTOR”.

The technology of eliminating a large amount of dust is disclosed in Korean Patent No. 10-1633878. The dust collector capable of eliminating fine dust is configured to include: a lower frame; a frame support mechanism being fixed by a fastening member to an upper portion of the lower frame, a filter housing being combined in such a manner as to ascend or descend within the frame support mechanism in a state of being supported by a guider; a filter plate being combined with the filter housing and filtering out dust particles; and a vibration generator being installed on the lower frame and applying vibration to the filter housing. In the dust collector, a large amount of fine dust adhered to a filter and particularly to a filter in use for a long time can be dusted off reliably and promptly with vibration causing the filter plate to ascend and descend.

However, the dust collector disclosed in Korean Patent No. 10-1633878 has a function of collecting dust, but has the limitation that people cannot use the dust collector while out of a house or an office or while on the move.

Recently, various attempts have been made to improve the human respiratory health in an environment where there is exposure to a variety of air pollutants. One example of these attempts is mask development.

Outside air is inhaled through the nose and mouth of the human respiratory system. Therefore, it is important to protect the nose and mouth with a mask to block air pollutants from being directly introduced into the human respiratory system.

The airtightness is the most important factor in the mask development.

That is, there is a need to improve the technology of preventing external harmful materials, such as fine dust and harmful gas, from being introduced into the mask.

However, the mask having the improved airtightness makes it difficult for a user of the mask to breathe.

Therefore, the mask needs to be developed in such a manner as to have a structure where the user's nose and mouth is not fully covered without making it difficult for the user of the mask to breathe. Furthermore, the mask needs to be developed in such a manner as to supply to the user only clean and safe air from which an external harmful material is filtered out.

A mask device in the related art that includes a fan and a filter purifies outside air and supplies the purified outside air to the user's nose and mouth, but cannot purify air exhaled from the user's nose and mouth. Thus, a virus can be spread to the outside while the user breathes. For this reason, the mask device in the related art is less effective in preventing a disease caused by the virus.

In addition, the mask device in the related art that includes the fan and the filter, when worn on the user's face, provides great mental discomfort.

That is, the worn mask device in the related art that includes the fan and the filter generates a large amount of noise while the fan is rotated by power of a motor. Furthermore, one portion of the mask device is brought into tight contact with the user's face, and thus drops of sweat are formed along an edge of the portion thereof. Due to the noise and the drops of sweat, the user feels a sense of discomfort.

For this reason, in the related art, attempts have been made to develop a mask device capable of providing purified air to the user's nose and mouth, purifying and discharging air exhaled from the user's nose and mouth, and thus maintaining a comfortably worn state. However, a satisfactory mask device has not yet been manufactured.

### DISCLOSURE

### Technical Problem

An objective of the present invention is to provide a mask device having an air curtain function, the mask device being configured to form an air curtain in the vicinity of a user's respirator organ, blocking introduction of contaminated outside air, blowing air purified through a built-in filter to the vicinity of the user's nose and mouth, and thus blocking fine dust.

Another object of the present invention is to provide a mask device having an airflow function, the mask device in which an LED sterilizer is installed to perform sterilization and purification at any time.

Still another object of the present invention is to provide a mask device capable of providing purified air to a user's nose and mouth, purifying and discharging air exhaled from the user's nose and mouth, and thus maintaining a comfortably worn state.

### Technical Solution

In order to achieve the above-described objectives, according to an aspect of the present invention, there is provided a mask device having an air curtain function, the mask device including: a main body brought into tight contact with the vicinities of a user's nose and mouth; a respirator installed in a lower portion of the main body for a user's respiration; and a cover being combined with a front surface of the main body and covering the main body and the respirator.

The respirator is configured to include a front-surface portion and a rear-surface portion.

The respirator includes an inhalation-air supply portion provided on an upper portion thereof and an air curtain portion provided under the inhalation-air supply portion.

The inhalation-air supply portion includes: an opening portion formed in the front-surface portion of the respirator; a cover portion in which a plurality of first vent holes through which outside air is introduced are formed, into which a filter is built, and which is separably mounted on the opening portion; a first air fan installed in a rear surface of the cover portion to introduce the outside air into the mask device; and an inhalation air hole portion formed in the rear-surface portion of the respirator in a manner that is positioned behind the first air fan.

The air curtain portion includes: an air introduction hole formed in the front-surface portion of the respirator; a plurality of air exhaust holes formed in a lower portion of the respirator; and a blower fan installed within the respirator to facilitate an airflow in the air curtain portion.

In the mask device, by operation of the blower fan, the outside air is introduced into the respirator through the air introduction hole, the introduced outside air is discharged to the outside through the air exhaust holes, and thus an air curtain is formed in a lower end portion of the respirator.

In the mask device, a plurality of exhalation vent air holes are additionally formed in the rear-surface portion of the respirator to facilitate discharging of the user's exhaled air.

In the mask device, the first air fan or the blower fan may be electrically connected to a charger.

In the mask device, the charger may be charged at any time through a charging terminal installed on one end of the respirator.

In the mask device, a tight-contact facial portion formed of a flexible material in such a manner as to be brought into tight contact with the user's face (or cheek) may be provided on an edge of a rear surface of the main body.

In the mask device, a plurality of vent air holes may be formed in a lower portion of the cover.

In the mask device, ear strings may be attached on both lateral edges, respectively, of the cover in such a manner that the user is able to wear the mask device by hanging the ear strings on the user's ears.

In the mask device, a plurality of sterilizing devices for sterilizing may be further installed on an internal wall of the main body.

In the mask device, an opening portion in the respirator may have a second threaded portion on an internal wall thereof.

In the mask device, a filter casing member may be provided in a manner that is combined in a nut-and-bolt manner with the second threaded portion.

In the mask device, the filter casing member may include a filter for filtering provided thereinto; a plurality of air holes through which to introduce the outside air being formed in a front surface thereof; and a first threaded portion formed on an external surface thereof in order to be combined with the second threaded portion.

In order to achieve the above-described objectives, according to another aspect of the present invention there is provided a fan rotation-type device having an air curtain function.

The fan rotation-type device includes a cover partially covering a user's face when the user hangs bands attached on both lateral edges of the cover on his/her ears, respectively; a main body that is combined with a rear surface of the cover and, by rotation of a fan, supplies to a user's nose and mouth outside air that is introduced through the cover and is filtered by a first filter; and a tight-contact member that is combined with a rear surface of the main body and, when an edge portion thereof is brought into tight contact with the vicinity of the user's nose and mouth, forms a partition space in front of the user's nose and mouth, wherein the main body includes; a first partition wall in which a first through-hole forcing the outside air introduced by the rotation of the fan to flow toward the user's nose and mouth in a distributed manner and a second through-hole forcing air exhaled from the user's nose and mouth to flow to the outside are formed; and a second filter being arranged outward from the first partition wall and filtering the air discharged through the second through-hole.

In the fan rotation-type mask device, the cover may include a netting portion in which a plurality of through-holes passing through the cover from a front surface thereof to a rear surface thereof are distributed.

In the fan rotation-type mask device, a diameter of the netting portion may be 30 to 70% of a height of the entire cover.

In the fan rotation-type mask device, the main body may include removable netting portions supporting the first filter and the second filter, respectively; and a motor being driven by power supplied from a battery and thus rotating the fan.

In the fan rotation-type mask device, the tight-contact portion may include a plurality of discharging holes running from within the partition space to a tight-contact member end portion brought into tight contact with the vicinity of the user's nose and mouth.

In the fan rotation-type mask device, the first through-hole in the first partition wall may be formed to have a greater open area than the second through-hole.

### Advantageous Effects

According to the present invention, an air curtain is formed in the vicinity of a user's respirator organ, introduction of contaminated outside air is blocked, and air purified through a built-in filter is blown to the vicinity of the user's nose and mouth. Thus, an advantage of blocking fine dust can be provided.

According to the present invention, at least one air fan is installed in a mask device, and thus air intake and the air exhaust are facilitated. Thus, an advantage of improving user convenience can be provided.

According to the present invention, an LED sterilizer is installed within the mask device. Thus, an advantage of performing sterilization and purification of the inside of the mask device at any time can be provided.

In addition, a main body of a fan rotation-type mask device according to the present invention includes a first partition wall and a second partition wall. Air exhaled from a user's nose and mouth that is discharged through the second filter in the first partition wall is filtered by the second filter. The air from which a foreign material is filtered out is discharged out of the main body. The foreign material, particularly, a virus, can be prevented from being discharged while the user breathes. Thus, an advantage of preventing the spread of the foreign material can be provided.

In addition, a diameter of a netting portion in a cover of the fan rotation-type mask device according to the present invention may be 30 to 70% of a height of the entire cover. The diameter of the netting portion can be maximized, and thus a diameter of a fan in the main body can be maximized. Accordingly, the occurrence of noise due to rotation of the fan can be minimized while outside air can be maximally prevented. Thus, a comfortably worn state can be maintained.

In addition, the fan rotation-type mask device according to the present invention includes a tight-contact member brought into tight contact with the vicinity of the user's nose and mouth. The tight-contact member includes a plurality of air holes. A part of the outside air introduced into a partition space formed by the tight-contact member is discharged through the air holes. Accordingly, ventilation takes place between the tight-contact member and the user's facial skin. Drops of sweat or the like can be prevented from being formed along an edge of the tight-contact member.

### DESCRIPTION OF DRAWINGS

FIG. 1 is a perspective view illustrating a mask device having an air curtain function according to a first embodiment of the present invention;

FIG. 2 is a perspective view illustrating the inside of the mask device having an air curtain function according to the first embodiment of the present invention;

FIG. 3 is an exploded perspective view illustrating the mask device having an air curtain function according to the first embodiment of the present invention;

FIG. 4 is a cross-sectional view taken along the forward-backward direction of the mask device having an air curtain function according to the first embodiment of the present invention;

FIG. 5 is a cross-sectional view taken along the upward-downward direction of the mask device having an air curtain function according to the first embodiment of the present invention;

FIG. 6 is a perspective view illustrating a mask device having an air curtain function according to a second embodiment of the present invention;

FIG. 7 is an exploded perspective view illustrating the mask device having an air curtain function according to the second embodiment of the present invention;

FIG. 8 is an exploded perspective exploded view illustrating a fan rotation-type mask device to a third embodiment of the present invention;

FIG. 9 is a view illustrating the assembled fan rotation-type mask device according to the third embodiment of the present invention;

FIG. 10 is a view illustrating a worn state of the fan rotation-type mask device according to the third embodiment of the present invention;

FIG. 11 is a vertical cross-sectional view taken along line VI-VI′ on the fan rotation-type mask device according to the third embodiment of the present invention in FIG. 9;

FIG. 12 is a view illustrating that air purified through the fan rotation mask device to according to the third embodiment of the present invention is discharged;

FIG. 13 is a view illustrating that outside air is discharged through air holes in a tight-contact member in the fan rotation-type mask device according to the third embodiment of the present invention; and

FIG. 14 is a horizontal cross-sectional view taken along line XIV-XIV′ on the fan rotation-type mask device according to the third embodiment of the present invention in FIG. 9.

### MODE FOR INVENTION

Mask devices having an air curtain function according to various embodiments of the present invention will be described in detail below with reference to the accompanying drawings.

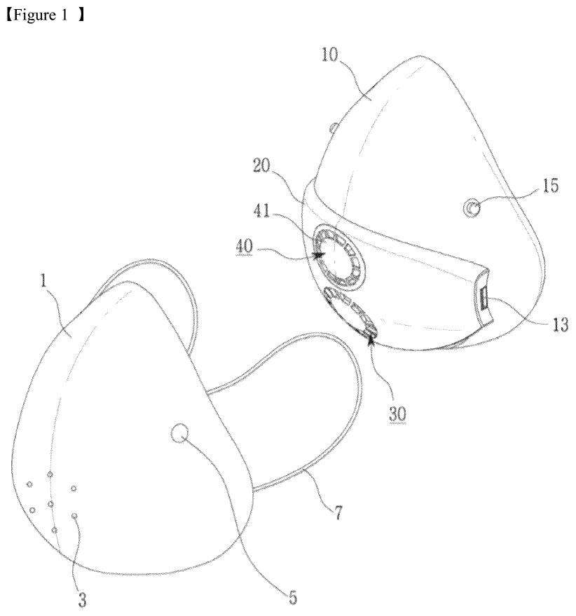

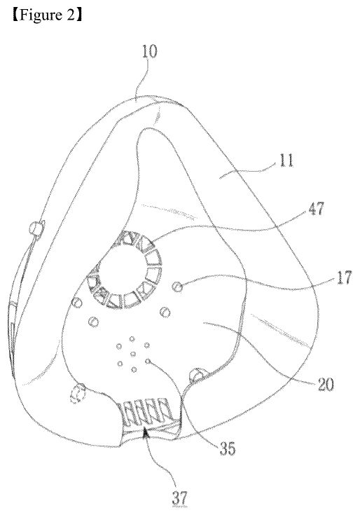

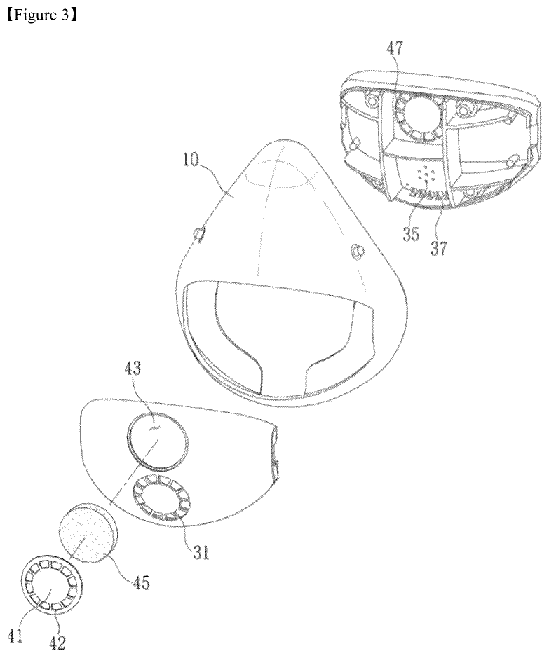

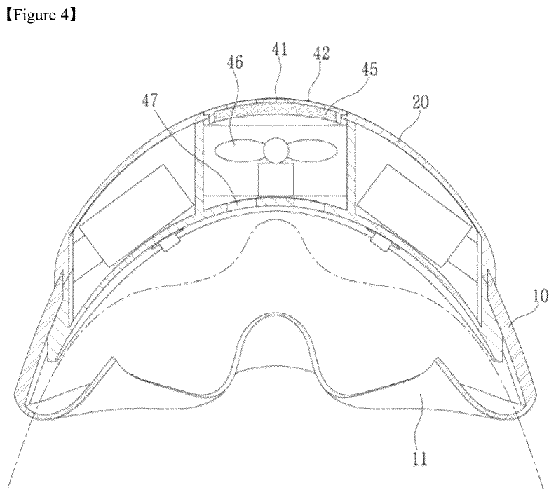

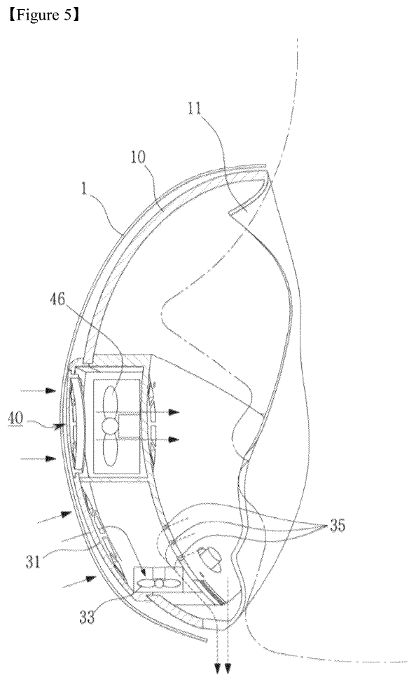

FIG. 1 is a perspective view illustrating a mask device having an air curtain function according to a first embodiment of the present invention. FIG. 2 is a perspective view illustrating the inside of the mask device having an air curtain function according to the first embodiment of the present invention. FIG. 3 is an exploded perspective view illustrating the mask device having an air curtain function according to the first embodiment of the present invention. FIG. 4 is a cross-sectional view taken along the forward-backward direction of the mask device having an air curtain function according to the first embodiment of the present invention. FIG. 5 is a cross-sectional view taken along the upward-downward direction of the mask device having an air curtain function according to the first embodiment of the present invention.

With reference to FIG. 1, the mask device according to the present invention has an air curtain function. An air curtain portion is installed on a lower portion of the mask device and can serve to initially block dust or foreign material from being introduced from the outside into the mask device. In addition, one or more air fans are installed in the mask device, and thus air intake and air exhaust are facilitated. Accordingly, user convenience can be greatly increased.

With reference to FIGS. 1 to 3, the mask device according to the present invention includes a main body 10, a respirator 20, and a cover 1. The main body 10 are brought into tight contact with the vicinities of a user's nose and mouth. For the user's respiration, the respirator 20 is installed on a lower portion of the main body 10. The cover 1 is combined with a front-surface portion of the main body and thus covers the main body 10 and the respirator 20.

It is desirable that the front-surface portion of the main body 10 is formed of a hard material in such a manner as to maintain a shape thereof. A tight-contact facial portion 11 is provided on an edge of a rear surface of the main body 10. The tight-contact facial portion 11 is formed of a flexible material in a such a manner as to be brought into tight contact with a user's face (or cheek).

The tight-contact facial portion 11 is formed of a flexible material in a manner that extends inward from the edge of the rear surface of the main body 10. Thus, the tight-contact facial portion 11 is brought into tight contact with the user's face and thus can prevent air or foreign material from being introduced into the mask device from the outside.

The main body 10 has a second combination means 15 on both upper end portions thereof. The second combination means 15 may be used when the cover 1 and the main body 10 are combined with each other.

The respirator 20 may be formed of a hard material in order to maintain a shape thereof and may be made up of a front-surface portion and a rear-surface portion. Accordingly, one or more air fans or charging devices for supplying air may be provided between the front-surface portion and the rear-surface portion.

The respirator 20 includes an inhalation-air supply portion 40 and has an air curtain portion 30 under the inhalation-air supply portion 40.

As implementation example, the inhalation-air supply portion 40 may be provided in an upper portion of the respirator 20 in such a manner that the inhalation-air supply portion 40 is positioned in the vicinities of the user's nose and mouth when the user wears the mask device.

The inhalation-air supply portion 40 has an opening portion 43 formed in the front-surface portion of the respirator 20. A cover portion 41 into which a filter 45 is built may be separably mounted on the opening portion 43. A plurality of first vent holes 42 are formed in the cover portion 41, and thus outside air may be introduced into the mask device. In addition, a first air fan 46 is installed behind the cover portion 41 to introduce the outside air into the mask device. An inhalation air hole portion 47 is formed in the rear-surface portion of the respirator 20 in a manner that is positioned behind the first air fan 46. The inhalation air hole portion 47 forces the outside air to flow, and thus the user can inhale the outside air.

In this case, the first air fan 46 is electrically connected to a charger. Thus, the charger may be turned on or off to operate the first air fan 46 or stop operating the first air fan 46. The user can selectively operate the charger. The charger may be charged at any time through a charging terminal 13 installed on one end of the respirator 20.

Therefore, by operation of the first air fan 46, the outside air is forced to be introduced through the first vent hole 42 in the cover portion 41 and then to pass through the filter 45. The filter 45 filters out a hazardous material or a foreign material from the outside air. The user can inhale the resulting purified outside air through the inhalation air hole portion 47.

The air curtain portion 30 is supplied with the outside air and guides the outside air to a lower portion of the respirator 20. Thus, the air curtain portion 30 can serve to smoothly discharge air forced out of the lung to the outside when the user exhales. At the same time, using this airflow, the air curtain portion 30 can serve to effectively block fine dust or a foreign material that may be introduced from the outside.

To this end, an air introduction hole 31 is formed in a front-surface portion of the air curtain portion 30 of the respirator 20, and a plurality of air exhaust hole 37 are formed in a lower end portion of the respirator 20. Moreover, a blower fan 33 is installed within the respirator 20. The blower fan 33 serves to force air to flow through the air curtain portion 30. In this case, the blower fan 33 is electrically connected to the charger. Thus, the charger may be turned on or off to operate the blower fan 33 or stop operating the blower fan 33. The user can selectively operate the charger. The charger may be charged at any time through the charging terminal 13 installed on one end of the respirator 20.

Therefore, the blower fan 33, when in operation, absorbs the outside air and the user's exhaled air. Then blower fan 33 discharges the outside air and the user's exhaled air to the outside through the air exhaust hole 37. Thus, air circulation can be facilitated, and at the same time, an air curtain effect can occur in the lower end portion of the respirator 20.

That is, the outside air is introduced into the respirator 20 through the air introduction hole 31, and the introduced outside air is discharged to the outside through the air exhaust hole 37. Thus, the air curtain effect can occur in the lower end portion of the respirator 20.

In addition, in order to facilitate the discharging of the user's exhaled air, a plurality of exhalation vent air holes 35 may be formed in the rear-surface portion of the respirator 20. In this case, likewise, when the user exhales, the exhaled air passes through the exhalation vent air hole 35, and then, by the operation of the blower fan 33, the exhaled air may be easily discharged to the outside through the air exhaust hole 37.

As an implementation example, the respirator 20 may include one or more partition walls that are arranged between the first air fan 46 and the blower fan 33 in such a manner that the first air fan 46 and the blower fan 33 are separated from each other in terms of determining an airflow direction.

The cover 1 may be formed of smooth cloth or leather. Thus cover 1 serves to cover the main body 10 for protection and at the same time to improve a design feature of the mask.

A plurality of air holes 3 are formed in a lower portion of the cover 1 and allows the outside air to be introduced into the respirator 20. In addition, the first combination means 5 is installed on one side of an internal surface of the cover 1. Thus, the first combination means 5 may be combined with the second combination means 15 installed on the main body 10.

In addition, ear strings 7 are attached on both lateral edges, respectively, of the cover 10. The user can wear the mask device by hanging the ear strings 7 on the user's ears.

In addition, a plurality of sterilizing devices 17 are installed on an internal wall of the main body 10. Thus, the inside of the mask device may be prevented from being contaminated. The sterilizing device 17 may be an LED emitting ultraviolet light.

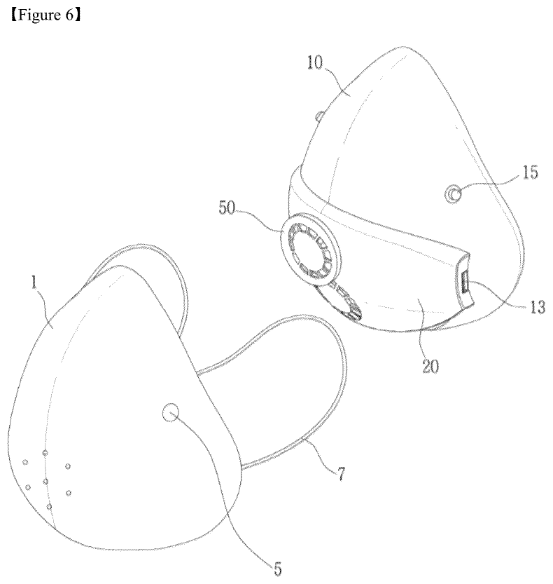

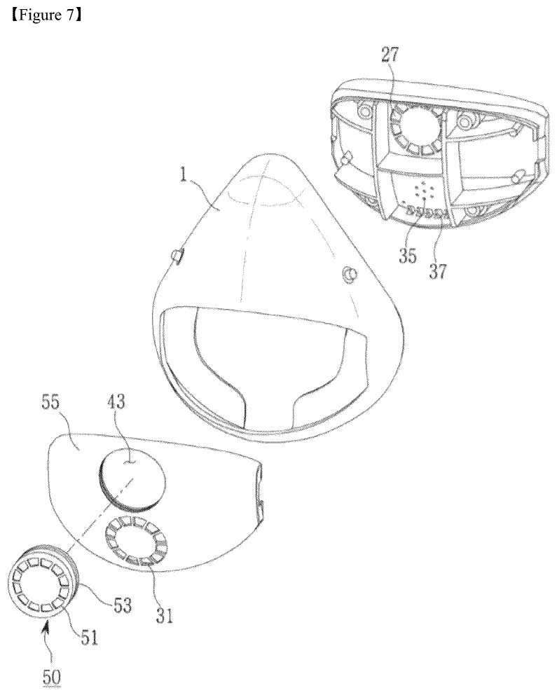

FIG. 6 is a perspective view illustrating a mask device having an air curtain function according to a second embodiment. FIG. 7 is an exploded perspective view illustrating the mask device having an air curtain function according to the second embodiment of the present invention.

With reference to FIGS. 6 and 7, the mask device having an air curtain function according to the second embodiment of the present invention is the same as or similar to the mask device having an air curtain function according to the first embodiment of the present invention in terms of configuration except that a filter casing member 50 substitutes for the cover portion 41. Accordingly, in a case where mask devices having an air curtain function according to various embodiment of the present invention have the same constituent element, a detailed description thereof is not repeated.

In the mask device according to the second embodiment of the present invention, the opening portion 43 formed in the front-surface portion of the respirator 20 includes a second threaded portion 55 on the internal wall thereof. The filter casing member 50 is combined in a nut-and-bolt manner with the second threaded portion 55.

The filter casing member 50 has a filter for filtering inside. A plurality of air holes 51 through which to introduce the outside air are formed in a front surface thereof, and a first threaded portion 53 is formed on an external surface thereof in a manner that is combined with the second threaded portion 55.

Therefore, the filter casing member 50 may be freely attached and detached to and from the respirator 20, and the filter may be easily replaced with or exchanged for a new one,

In addition, a plurality of sterilizing and disinfecting devices for sterilization may be further installed in the vicinity of the filter casing member 50 (in the vicinity of the opening portion 43). Accordingly, the filter within the filter casing member may be sterilized and disinfected. Moreover, the effect of eliminating a virus not filtered out by the filter can be achieved.

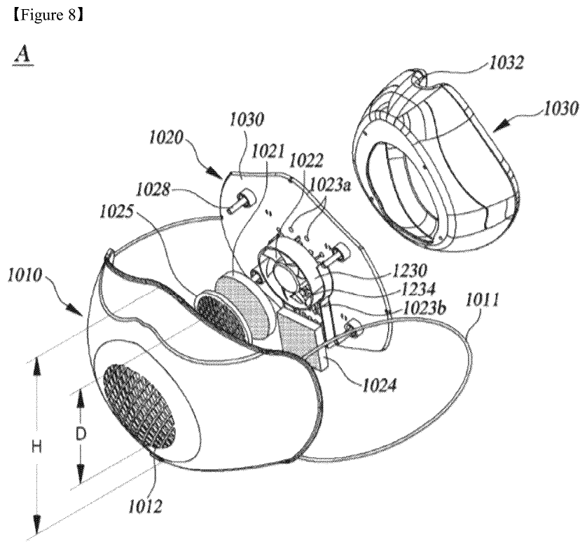

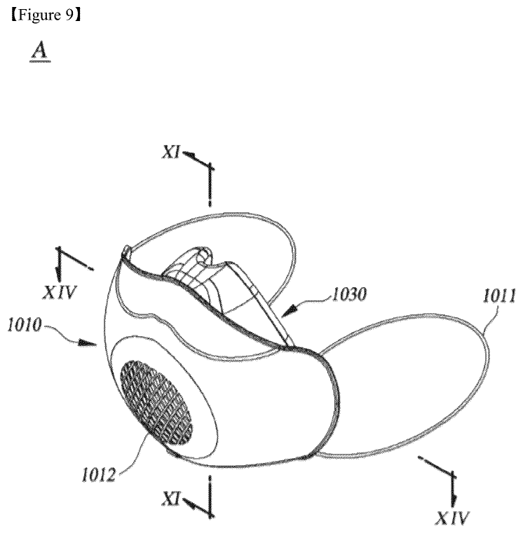

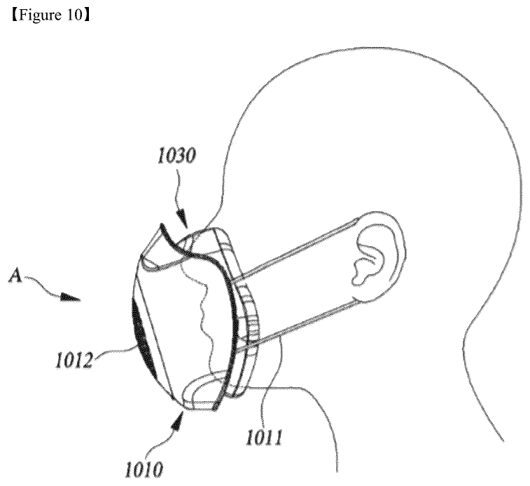

As illustrated in FIGS. 8 to 10, a fan rotation-type mask device A according to a third embodiment of the present invention includes a cover 1010, a main body 1020, and a tight-contact member 1030.

When bands 1011 attached on both lateral edges of the cover 1010 are hung on the user's ears, respectively, the cover 1010 according to the present invention partially covers the user's face.

Accordingly, with the cover 1010, the fan rotation-type mask device A according to the present invention is worn on the user's face.

In this case, the cover 1010 includes a netting portion 1012 that has a plurality of through-holes (not designated by a reference numeral) passing therethrough from a front surface thereof to a rear surface thereof. Thus, through the netting portion 1012, the outside air may be introduced toward the main body 1020 positioned behind the cover 1010.

The netting portion 1012 here has a diameter D that is 30 to 70% of a height H of the entire cover 1010. The fan 1022 having a diameter corresponding to the diameter D of the netting portion 1012 is provided to the main body 1020, and thus the diameter of the fan 1022 may be maximized. Accordingly, an outside-air introduction path may be expanded, and thus the occurrence of noise due to rotation of the fan 1022 can be maximally prevented while the outside air is introduced.

However, in a case where the diameter D of the netting portion 1012 is less than 30% of the height H of the entire cover 1010, the effect of expanding the outside-air introduction path may be insignificant. Furthermore, in a case where the diameter D of the netting portion 1012 is greater than 70% of the height H of the entire cover 1010, the size of the fan 1022 is increased, and thus the thickness and the like thereof are also excessively increased. Therefore, it is difficult to miniaturize the fan rotation-type mask device A according to the present invention. For this reason, it is desirable that the diameter D of the netting portion 1012 is 30 to 70% of the height H of the entire cover 1010.

The cover 1010 includes a combination means 1013 installed on a rear surface thereof. Accordingly, the combination means 1013 of the cover 1010 is combined with a combination means 1028 described below on a front surface of the main body 1020, and thus the cover 1010 and the main body 1020 are combined with each other.

In this case, the combination means 1013 of the cover 1010, if freely combinable and separable with and from the combination means 1028 of the main body 1020, is not limited to any normal structure and combination method. Therefore, a detailed description of the combination means 1013 is omitted.

The main body 1020 according to the present invention is combined with a rear surface of the cover 1010. By the rotation of the fan 1022, the main body 1020 supplies to the user's nose and mouth the outside air that is introduced through the cover 1010 and is filtered by a first filter 1021.

Therefore, the user who wears the fan rotation-type mask device A according to the present invention inhales the purified air supplied by the main body 1020 through his/her nose and mouth.

In this case, the main body 1020 includes a first partition wall 1023 in which a first through-hole 1023a and a second through-hole 1023b are formed. The first through-hole 1023a forces the outside air flowing by the rotation of the fan 1022 to flow in a distributed manner toward the user's nose and mouth. The second through-hole 1023b forces the air exhaled from the user's nose and mouth to flow to the outside in a distributed manner. In the manner, through the first through-hole 1023a and the second through-hole 1023b in the first partition wall 1023, the outside air can be smoothly supplied, and the air can be smoothly exhaled from the user's nose and mouth.

In this case, it is desirable that the first through-hole 1023a in the first partition wall 1023 is formed to have a greater entire open area than the second through-hole 1023b. Thus, the flow of the outside air in a distributed manner after passing through the first through-hole 1023a may be better facilitated.

The main body 1020 includes a second filter 1024 that is arranged outward from the first partition wall 1023 and filters the air discharged through the second through-hole 1023b. The second filter 1024 may filter out a foreign material from the air exhaled from the user's nose and mouth.

The main body 1020 includes a removable netting body 1025 supporting the first filter 1021. With the removable netting body 1025, not only can the first filter 1021 be prevented from being deviated, but the outside air can also be introduced toward the first filter 1021.

Although not illustrated in FIG. 8, the main body 1020 may further include a netting body (not illustrated) supporting the second filter 1024. In this case, the netting body supporting the second filter 1024 may employ any shape suitable to support the second filter 1024.

In this case, the netting body 1025 is formed in a manner that is removably attached. Thus, the netting body 1025 is separable from the main body 1020. Accordingly, the first filter 1021 and the second filter 1024 may be exchanged with new ones.

The main body 1020 includes a motor 1026 driven by power supplied from a battery 1027. By the driving of the motor 1026, the fan 1022 may be rotated.

In this case, the battery 1027 is a chargeable battery and may be reused by charging. For charging, the battery 1027 is connected to a charging terminal (not illustrated), and a power cable (not illustrated) is connected to the charging terminal.

The first filter 1021 and the second filter 1024 of the main body 1020, if it can filter out a foreign material contained in the outside air, for example, ultra-fine dust or a virus, are not limited to any structure and filtering method. One example thereof is a HEPA filter.

The first filter 1021 and the second filter 1024 are installed in opening portions (not designated by a reference numeral), respectively, in the main body 1020. Ventilation through the first filter 1021 and the second filter 1024 may take place.

The main body 1020 includes the combination means 1028 installed on the front surface thereof. The combination means 1028 of the main body 1020 is combined with the combination means 1013 of the cover 1010, and thus the main body 1020 is combined with the cover 1010.

In this case, the combination means 1028 of the main body 1020, if freely combinable or separable with and from the combination means 1013 of the cover 1010, is not limited to any structure and combination method. Therefore, a description of the combination means 1028 is omitted.

The main body 1020 may further include a fan accommodation unit 1230 that is formed in such a manner as to accommodate a fan and is attached or combined to and with the first partition wall 1023. In this case, the fan accommodation unit 1230, although illustrated as having a shape of a hollow circular cylinder, is not necessarily limited to this shape. The fan accommodation unit 1230 may have a shape of a hollow polyprism. The fan accommodation unit 1230 includes a vent hole 1234 formed in the lower portion thereof.

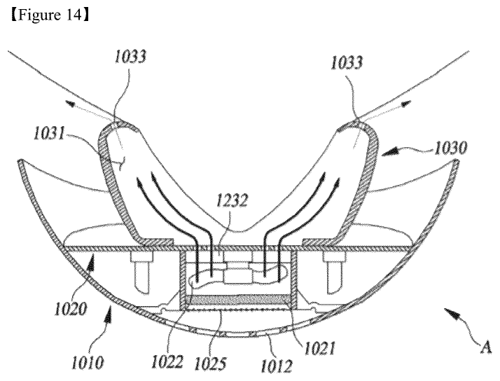

The vent hole 1234 is formed in such a manner that at least one part of the outside air introduced by the fan 1022 flows toward the second filter 1024. The vent hole 1234, although illustrated as having a height that is approximately half of a height of the fan accommodation unit 1230, is not necessarily limited to this height. For example, the vent hole 1234 may be formed in a manner that has the same height as the fan accommodation unit 1230. In other words, the fan accommodation unit 1230 may be formed in such a manner that at least one portion of an external circumferential surface thereof is discontinuous. The tight-contact member 1030 according to the present invention is combined with a rear surface of the main body 1020, and an edge portion of the tight-contact member 1030 is brought into tight contact with the vicinity of the user's nose and mouth. Thus, a partition space 1031 (refer to FIG. 14) is formed in front of the user's nose and mouth.

The tight contact of the tight-contact member 1030 with the user's nose and mouth prevents the outside air from being introduced between the main body 1020 and the user's facial skin and prevents the air exhaled from the user's nose and mouth from leaking.

In this case, a depression portion 1032 having a curved shape is formed in a manner that is recessed into an upper end portion of the tight-contact member 1030. The user's nasal bridge is inserted into the depression portion 1032 in a state where the fan rotation-type mask device A according to the present invention is worn on the user's face. Thus, the tight-contact member 1030 is prevented from being raised from the user's facial skin due to the shape of the user's nose and thus can be brought into tight contact with the user's facial skin.

The tight-contact member 1030 includes a plurality of air holes 1033 (refer to FIG. 14). The plurality of air holes 1033 run from within the partition space 1031 to a tight-contact member 1030 end portion brought into tight contact with the vicinity of the user's nose and mouth. Thus, one part of the outside air introduced into the partition space 1031 is discharged through the plurality of air holes 1033. Accordingly, ventilation takes place between the tight contact member 1030 and the user's facial skin.

The tight-contact member 1030 is formed of a flexible material, for example, silicone. Thus, the tight-contact member 1030 is easily curved along the user's face. Accordingly, the tight-contact member 1030 can be brought into tight contact with the user's nasal skin, particularly, with the vicinity of the user's nose and mouth in a more airtight manner.

The fan rotation-type mask device A according to the third embodiment of the present invention is capable of inhaling purified air. The inhalation of the purified air will be described in detail below.

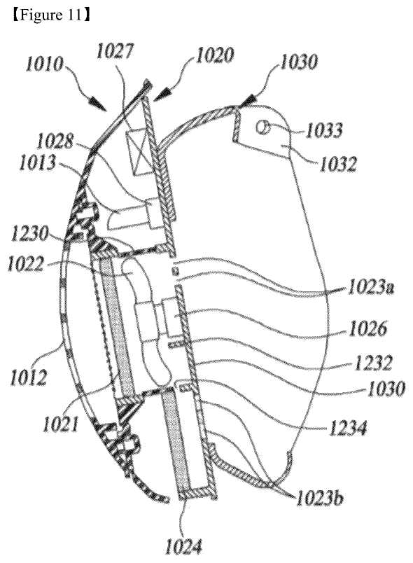

As illustrated in FIG. 11, the user wears the fan rotation-type mask device A according to the present invention in a manner that covers his/her face, more particularly, his/her mouth and nose.

In this case, a netting portion 1012 is formed in the cover 1010. The outside air is introduced from the outside through the netting portion 1012 in the cover 1010 and to the main body 1020 combined with the rear surface of the cover 1010.

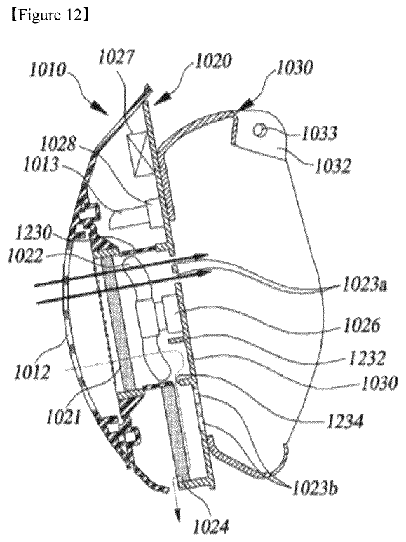

The main body 1020 includes the first filter 1021 and the fan 1022. As illustrated in FIG. 12, by the rotation of the fan 1022, the outside air is forced to flow from the cover 1010 through the first filter 1021 into the main body 1020. The first filter 1021 may filter out a foreign material, for example, ultra-fine dust or a virus from the outside air.

At this point, the outside air from which the first filter 1021 filters out the foreign material is forced to flow in a distributed manner by the first through-hole 1023a formed in the first partition wall 1023 of the main body 1020. Thus, the outside air naturally flows to the user's nose and mouth, and the user's nose and mouth can inhale the purified air. Accordingly, the foreign material contained in the outside air can be prevented from being inhaled, thereby eliminating a respiratory disease.

However, when a gap occurs between the main body 1020 and the user's facial skin, the outside air containing the foreign material can be introduced through the gap.

The tight-contact member 1030 included in the fan rotation-type mask device A according to the present invention is combined with the rear surface of the main body 1020. The edge portion of the tight-contact member 1030 are brought into tight contact with the vicinity of the user's nose and mouth, and thus the outside air containing the foreign material can be prevented from being introduced between the main body 1020 and the user's facial skin. In this case, the tight-contact member 1030 is formed of a flexible material. Thus, the tight-contact member 1030 is easily curved along the user's facial skin. Accordingly, the tight-contact member 1030 can be brought into tight contact with the user's face, more particularly, with the vicinity of the user's nose and mouth. The depression portion 1032 having a curved shape is formed in the upper end portion of the tight-contact member 1030. The user's nasal bridge is inserted into the depression portion 1032. Accordingly, the tight-contact member 1030 can be prevented from being raised from the user's facial skin and thus can be brought into tight contact with the user's facial skin in a more airtight manner.

In addition, the fan accommodation unit 1230 according to the present invention may further include a second partition wall 1232. The second partition wall 1232 protrudes from the first partition wall 1023 in such a manner as to face the fan 1022 and extends in a diameter direction of the fan accommodation unit 1230. At least one part of the outside air that is introduced by the fan 1022 and stays in a space formed between the fan 1022 and the first partition wall 1023 may not flow to the first through-hole 1023a because of the second partition wall 1232. The space formed between the fan 1022 and the first partition wall 1023 within the fan accommodation unit 1230 may be divided by the second partition wall 1232 into an upper space and a lower space in terms of determining the airflow direction.

FIG. 12 is a view illustrating that the purified air is inhaled through the fan rotation-type mask device according to the third embodiment of the present invention. In FIG. 12, a direction of a primary airflow is indicated by a thick arrow, and a direction of a secondary airflow is indicated by a narrow arrow.

As indicated by the thick arrow in FIG. 12, at the time of the air intake, the driving of the fan 1022 forces the outside air to flow into the main body 1020.

As indicated by the narrow arrow in FIG. 12, at the time of the air intake, at least one part of the air introduced into the main body 1020 is discharged through the vent hole 1234 to below the main body 1020. Therefore, the air curtain effect can occur in the lower portion of the main body 1020. When an air curtain is formed, contaminated outside air can be prevented from being introduced into the fan rotation-type mask device A. In this case, the air discharged to the vent hole 1234 does not flow toward the first through-hole 1023a because of the second partition wall 1232, and thus the air curtain can be formed in a more effective manner.

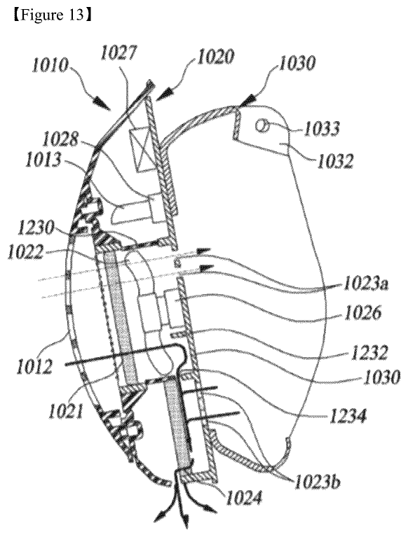

FIG. 13 is a view illustrating that the purified air is discharged through the fan rotation-type mask device according to the third embodiment of the present invention. In FIG. 13, a direction of a primary airflow is indicated by a thick arrow, and a direction of a secondary airflow is indicated by a narrow arrow.

As indicated by the thick arrow in FIG. 13, at the time of the air exhaust, at least one part of the outside air introduced by the fan 1022 passes through the vent hole 1234. In addition, the user's exhaled air and the introduced outside air join together and are discharged out of the mask device A. While this discharging takes place, the air curtain may be formed. As is the case with the air intake, air discharged to the vent hole 1234 does not flow toward the first through-hole 1023a because of the second partition wall 1232. Accordingly, the air curtain may be formed in a more effective manner.

As indicated by the narrow arrow in FIG. 0.13, at the time of the air exhaust, at least one other part of the outside air introduced by the fan 1022 may be introduced into the tight-contact member 1030. It is desirable that the fan 1022 is driven with less power at the time of the air exhaust than at the time of the air intake.

The fan rotation-type mask device A according to the present invention may discharge the purified air. The discharging of the purified air will be described in detail below.

The main body 1020 according to the present invention includes the second filter 1024 arranged in front of the second through-hole 1023b in the first partition wall 1023. The second filter 1024 filters the air that is exhaled from the user's nose and mouth and passes through the second through-hole 1023b. The exhaled air passing through the second filter 1024 in a distributed manner to the outside. Accordingly, a foreign material, for example, a virus, can be filtered out from the air exhaled from the user's nose and mouth. Therefore, while the user breathes, particularly, while the user exhales, a virus cannot be transmitted to the outside, thereby preventing the spread of the virus.

In addition, the fan rotation-type mask device A according to the present invention may maintain a comfortably worn state. The comfortably worn state is described in detail below.

The netting portion 1012 in the cover 1010 may have the diameter D that is 30 to 70% of the height H of the entire cover 1010. The fan 1022 having a diameter corresponding to the diameter D of the netting portion 1012 is provided to the main body 1020, and thus the diameter of the fan 1022 may be maximized. Accordingly, the outside-air introduction path may be expanded, and thus the occurrence of noise due to rotation of the fan 1022 can be maximally prevented while the outside air is introduced. This reduction in noise can contribute to maintaining the comfortably worn state.

Moreover, according to the present invention, the tight-contact member 1030 includes the plurality of air holes 1033 running from within the partition space 1031 to the tight-contact member 1030 end portion brought into tight contact with the vicinity of the user's nose and mouth. Thus, as illustrated in FIG. 14, one part of the outside air introduced into the partition space 1031 is discharged through the plurality of air holes 1033. Accordingly, ventilation takes place between the tight contact member 1030 and the user's facial skin. Thus, drops of sweat or the like can be prevented from being formed along an edge of the tight-contact member 1030. This ventilation can contribute to maintaining the comfortably worn state.

Constituent elements illustrated in the drawings and described in the present specification are only exemplary ones according to the most preferred embodiments of the present invention and do not represent practical applications of the technical idea of the present invention in its entirety, and therefore it should be apparently understood that, as substitutions for these constituent elements, various equivalents and modifications thereto would be available at the time of filing the present patent application.

### CROSS-REFERENCE TO RELATED APPLICATION

The present application claims priority to Korean Patent Application No. 10-2020-0031878, filed Mar. 16, 2020, the entire contents of which is incorporated herein for all purposes by this reference under 35 U.S.C. § 119(a). Moreover, under the relevant patent laws of corresponding countries other than the United States of America, the present application also claims priority to Korean Patent Application No. 10-2020-0031878, filed Mar. 16, 2020, the entire contents of which is incorporated herein for all purposes by this reference.

### DESCRIPTIONS OF REFERENCE NUMERAL

1: Cover 3: Vent air hole
5: First combination means 7: Ear string
10: Main body 11: Tight-contact facial portion
15: Second combination means 17: Sterilizing device
20; Respirator 30: Air curtain portion
31: Air introduction hole 33: Blower fan
35: Exhalation vent air hole 37: Air exhaust hole
40: Inhalation-air supply portion 41: Cover portion
42: First vent hole 43: Opening portion
45: Filter 47: Inhalation-air hole portion
50: Filter casing member 51: Air hole
53: First threaded portion 55: Second threaded portion
1010: Cover 1011: Band
1012: Netting portion 1013: Combination means
1020: Main body 1021: First filter
1022: Fan 1023: First partition wall
1023a: First through-hole 1023b: Second through-hole
1024: Second filter 1025: Netting portion
1026: Motor 1027: Battery
1028: Combination means 1030: Tight-contact member
1031: Partition space 1032: Depression portion
1033: Air hole 1230: Fan accommodation portion
1232: Second partition wall 1234: Vent hole
A: Mask device D: Diameter of netting portion
H: Height of entire cover

1. A mask device having an air curtain function, the mask device comprising:
a main body brought into tight contact with the vicinities of a user's nose and mouth; and
a respirator installed on the main body for a user's respiration,
wherein the respirator comprises:
an inhalation-air supply portion; and
an air curtain portion provided under the inhalation-air supply portion,
wherein the inhalation-air supply portion comprises:
an opening portion formed in a front-surface portion of the respirator;
a cover portion in which a plurality of first vent holes through which outside air is introduced are formed, into which a filter is built, and which is separably mounted on the opening portion;
a first air fan installed in a rear surface of the cover portion to introduce the outside air into the mask device; and
an inhalation air hole portion formed in a rear-surface portion of the respirator in a manner that is positioned behind the first air fan,
wherein the air curtain portion comprises:
an air introduction hole formed in the front-surface portion of the respirator;
a plurality of air exhaust holes formed in a lower portion of the respirator; and
a blower fan installed within the respirator to facilitate an airflow in the air curtain portion,
wherein by operation of the blower fan, the outside air is introduced into the respirator through the air introduction hole, the introduced outside air is discharged to the outside through the air exhaust holes, and thus an air curtain is formed in a lower end portion of the respirator, and
wherein a plurality of exhalation vent air holes are additionally formed in the rear-surface portion of the respirator to facilitate discharging of the user's exhaled air, and the blower fan is provided under the air introduction hole and the exhalation vent air holes.

2. The mask device of claim 1, wherein the first air fan or the blower fan is electrically connected to a charger, and the charger is charged at any time through a charging terminal installed on one end of the respirator.

3. The mask device of claim 1, wherein a tight-contact facial portion formed of a flexible material in such a manner as to be brought into tight contact with the user's face (or cheek) is provided on an edge of a rear surface of the main body.

4. The mask device of claim 1, further comprising:
a cover being combined with a front surface of the main body and covering the main body and the respirator,
wherein a plurality of vent air holes are formed in a lower portion of the cover, and ear strings are attached on both lateral edges, respectively, of the cover in such a manner that the user is able to wear the mask device by hanging the ear strings on the user's ears.

5. The mask device of claim 1, wherein a plurality of sterilizers for sterilization are further installed on an internal wall of the main body.

6. The mask device of claim 1, wherein an opening portion in the respirator has a second threaded portion on an internal wall thereof, and a filter casing member is provided in a manner that is combined in a nut-and-bolt manner with the second threaded portion.

7. The mask device of claim 6, wherein the filter casing member comprises:
a filter for filtering provided thereinto;
a plurality of air holes through which to introduce the outside air being formed in a front surface thereof; and
a first threaded portion formed on an external surface thereof in order to be combined with the second threaded portion.

8. The mask device of claim 7, wherein a plurality of sterilizing and disinfecting devices for sterilization are further installed in the vicinity of the filter casing member.

9. A mask device having an air curtain function, the mask device comprising:
a main body at least one portion of which is formed in such a manner as to be brought into tight contact with the vicinities of a user's nose and mouth;
a respirator installed on a front surface of the main body;
an inhalation-air supply portion provided in the respirator in a manner that is arranged in the vicinities of the user's nose and mouth when a user wears the mask device; and
an air curtain portion provided in the respirator in a manner that is arranged under the inhalation-air supply portion,
wherein the inhalation-air supply portion comprises a first air fan installed within the respirator, the air curtain portion comprises a blower fan installed within the respirator, and the respirator comprises at least one partition wall that is formed to be arranged between the first air fan and the blower fan and thus separates the first air fan and the blower fan from each other in terms of determining an airflow direction.

10. A fan rotation-type mask device, comprising:
a main body that, by rotation of a fan, supplies to a user's nose and mouth outside air that is introduced and is filtered by a first filter; and
a tight-contact member that is combined with a rear surface of the main body and, when an edge portion thereof is brought into tight contact with the vicinity of the user's nose and mouth, forms a partition space in front of the user's nose and mouth,
wherein the main body comprises;
a first partition wall in which a first through-hole forcing the outside air introduced by the rotation of the fan to flow toward the user's nose and mouth in a distributed manner and a second through-hole forcing air exhaled from the user's nose and mouth to flow to the outside are formed; and
a second filter being arranged outward from the first partition wall and filtering the air discharged through the second through-hole.

11. The fan rotation-type mask device of claim 10, further comprising:
a cover partially covering the user's face.

12. The fan rotation-type mask device of claim 11, wherein the cover comprises:
a netting portion in which a plurality of through-holes passing through the cover from a front surface thereof to a rear surface thereof are distributed.

13. The fan rotation-type mask device of claim 12, wherein a diameter of the netting portion is 30 to 70% of a height of the entire cover.

14. The fan rotation-type mask device of claim 10, wherein the main body comprises:
one or more removable netting portions supporting the first filter and the second filter, respectively; and
a motor being driven by power supplied from a battery and thus rotating the fan.

15. The fan rotation-type mask device of claim 10, wherein the tight-contact portion comprises:
a plurality of discharging holes running from within the partition space to a tight-contact member end portion brought into tight contact with the vicinity of the user's nose and mouth.

16. The fan rotation-type mask device of claim 11, wherein an entire open area of the first through-hole in the first partition wall is greater than an open area of the second through-hole.

17. The fan rotation-type mask device of claim 10, wherein the main body further comprises:
a fan accommodation portion in the shape of a hollow cylinder which is formed in such a manner as to accommodate the fan, which is attached to the first partition wall, and in which a vent hole is formed, the vent hole forcing one part of the outside air introduced by the fan to flow toward the second filter.

18. The fan rotation-type mask device of claim 17, wherein the fan accommodation portion further comprises a second partition wall that is formed to protrude from the first partition wall in a manner that faces the fan and prevents at least one part of the outside air introduced by the fan from flowing to the first through-hole.
